(19) United States
(12) Patent Application Publication
(10) Pub. No.: US 2016/0135828 A1
(43) Pub. Date: May 19, 2016

(54) SHOCK WAVE VALVULOPLASTY DEVICE AND METHODS

(71) Applicant: SHOCKWAVE MEDICAL, INC., Fremont, CA (US)

(72) Inventors: Daniel HAWKINS, Fremont, CA (US); Mark C. T. HUANG, Pleasanton, CA (US); Show-Mean Steve WU, Fremont, CA (US); Arnel CASTRO, Santa Clara, CA (US); Rainier BETELIA, San Jose, CA (US); Adam R. TANNER, Campbell, CA (US)

(21) Appl. No.: 14/940,029

(22) Filed: Nov. 12, 2015

Related U.S. Application Data
(60) Provisional application No. 62/080,131, filed on Nov. 14, 2014.

(51) Int. Cl. A61B 17/22

(52) U.S. Cl. CPC A61B 17/22022; A61B 2017/22025; A61B 2017/22062; A61B 2017/22098; A61B 2017/00243

(57) ABSTRACT

Described here are devices and methods that may facilitate treatment of a calcified heart valve with shock waves. A shock wave valvuloplasty device may be advanced through a patient's vasculature and self-align with cusps of a calcified aortic valve. Alignment may be facilitated by a central anchor and/or U-shaped distal bends of positioning wires. An elongated carrier may be slidably disposed over a portion of the positioning wire and carry one or more electrode assemblies that may generate shock waves. An inflatable balloon may sealably enclose the electrode assembles, and shock waves may propagate through the liquid-filled balloon and transfer energy to adjacent calcified valve cusps. The shock wave valvuloplasty device may comprise one or more features to direct shock waves to specific areas of calcified tissue. Some variations also comprise a central anchor configured to be positioned below the valve leaflets.

### CROSS-REFERENCE TO RELATED APPLICATIONS

This application claims priority to U.S. Provisional Application Ser. No. 62/080,131, filed Nov. 14, 2014, which is hereby incorporated by reference in its entirety.

### BACKGROUND

Aortic calcification, also called aortic sclerosis, is a buildup of calcium deposits on the aortic valve in the heart. In some cases, the bulk of calcium deposits may be great enough to cause aortic stenosis, or narrowing of the opening of the aortic valve. Aortic stenosis impairs blood flow through the valve, which can lead to heart failure, chest pain, loss of consciousness, or death. In some cases, development of aortic stenosis may be accelerated by a congenital defect resulting in the aortic valve having two cusps (bicuspid) instead of three (tricuspid). If the valve opening becomes severely narrowed, aortic valve replacement surgery may be necessary. Alternatively, the aortic valve area can be opened or enlarged with a more tolerable procedure, such as expanding a balloon in the valve opening (balloon valvuloplasty). This may temporarily improve symptoms in high-risk patients who are not candidates for valve replacement surgery or act as a bridge to valve replacement surgery. However, in addition to the valve opening renarrowing after balloon valvuloplasty, the tremendous amount of energy in the balloon that is transferred to a valve may result in trauma to the valve.

An alternative method for treating calcified aortic valves has been proposed based on electrohydraulic lithotripsy. Electrohydraulic lithotripsy has typically been used for breaking calcified deposits or “stones” in the urinary or biliary tracts. It has recently been shown that lithotripsy electrodes may similarly be useful for breaking calcified plaques in arteries. Shock waves generated by lithotripsy electrodes may be used to controllably fracture a calcified lesion and prevent the sudden stress and injury to a valve that can occur during balloon valvuloplasty. Systems for using this treatment strategy have been described, for example, in U.S. Pat. Publ. No. 2014/0046353, U.S. Pat. Publ. No. 2010/0114020, and U.S. Pat. No. 8,574,247. Generally, in the embodiments described therein, a balloon is placed adjacent to calcified tissue, and the balloon is inflated with a liquid. Within a balloon is a shock wave generator that produces shock waves that propagate through the liquid and transfer mechanical energy to the adjacent calcified tissue. The shock waves break apart or otherwise disrupt calcified plaques, which may allow cusps of a valve to move more freely and enlarge the valve opening area. Another benefit of these devices may be their ability to prepare a valve area for transarterial aortic valve replacement where the aortic valve is replaced with an expandable valve structure. Such procedures benefit from a smooth, non-calcified aortic circumference to attach the new valve. Shock waves may break apart calcium deposits on the valve and aortic wall to provide a smoother, more pliable valve bed to attach an expandable valve. Specialized electrodes have been described to function as shock wave generators at a distal end of a catheter, such as in U.S. Pat. Publ. No. 2014/0243820. An embodiment of shock wave generator described in U.S. Pat. Publ. No. 2014/0052147 comprises flat electrodes that may be positioned around or on the side of a catheter, which may facilitate propagation of shock waves outward from the side of a catheter.

A shock wave may propagate in a specific direction from its source. Shock waves directed at calcified tissue may transfer more energy to the calcified tissue than shock waves directed away from the calcified tissue. In addition, the closer a shock wave generator is to calcified tissue, the more efficient the transfer of energy is from shock waves to the calcified tissue. The present invention provides improvements to previously described devices by providing a means of directing shock waves to calcified valve tissue and delivering energy from the shock waves to the calcified valve tissue in the most efficient way possible.

### BRIEF SUMMARY

Described herein are devices and methods that may facilitate treatment of a calcified heart valve with shock waves. A shock wave valvuloplasty device may be advanced through a patient's vasculature and self-align with cusps of a calcified aortic valve. Alignment may be facilitated by a central anchor and/or U-shaped distal bends of positioning wires. An elongated carrier may be slidably disposed over a portion of the positioning wire and carry one or more electrode assemblies that may generate shock waves. An inflatable balloon may sealably enclose the electrode assembles, and shock waves may propagate through the liquid-filled balloon and transfer energy to adjacent calcified valve cusps. The shock wave valvuloplasty device may comprise one or more features to direct shock waves to specific areas of calcified tissue.

One variation of a valvuloplasty device may comprise a central tubular member carrying at least two balloon catheters, each balloon catheter including a central carrier having a shock wave generator, each balloon catheter further including a balloon affixed to the distal end of the carrier, and a central anchor extending between and beyond the ends of the balloons and configured to pass through the aortic leaflets and into the ventricle to stabilize the position of the balloon catheters. The balloon may be inflatable with a conductive fluid, and activation of each of the shock wave generators may create a shock wave that propagates through the associated balloon. In some variations, a length of the wire may extend within the interior of the balloon, and the carrier is slidable over the wire within the balloon such that sliding the carrier over the wire changes the location of the shock wave generator. The length of the wire within the balloon may have a bend and the carrier may be slidable over the bend. In some variations, the central anchor may comprise an inflatable member disposed over a distal portion of the central tubular member. A valvuloplasty device may comprise a flow diverter disposed over the distal portion of the central tubular member. The flow diverter may comprise a proximal opening, a distal opening, and a lumen extending therebetween, and the flow diverter may be arranged such that the proximal and distal openings are located outside of the inflatable member. In other variations, the central anchor may be a self-expanding anchor. In some variations, the central anchor comprises a compressible scaffold structure or a compressible cage. The shock wave generator of each of the balloon catheters may comprise a pair of electrodes arranged such that when said pair of electrodes is connected to a high voltage source, a plasma arc is created across the electrodes resulting in a shock wave that propagates through the associated balloon. Alternatively or additionally, the shock wave generator of each of the balloon catheters may comprise a laser light source. More generally, the central anchor may comprise a shape-memory material.

Another variation of a valvuloplasty device may comprise an elongated hollow carrier, said carrier including at least one pair of electrodes, a balloon affixed to the distal end of the carrier, said balloon being inflatable with a conductive fluid, and where when said pair of electrodes are connected to a high voltage source, a plasma arc is created across the electrodes resulting in a shock wave that propagates through the balloon. The electrodes may be located within the balloon. The device may also comprise a positioning wire slidably received within the carrier, said positioning wire having a bend formed along a length of the wire that is located within the balloon and arranged so that when the position of the carrier is adjusted with respect to the positioning wire, the bend in the wire varies the angular orientation of the pair of electrodes to adjust the propagation direction of the shock wave. The positioning wire may extend from the distal end of the balloon, and may include a U-shaped section configured to center and position the balloon within a cusp of a valve leaflet. Some variations may also comprise an elongate member and an anchor disposed over a distal portion of the elongate member. The elongate member may be configured to extend distally beyond the balloon to pass the anchor through a valve orifice to stabilize the position of the balloon. In some variations, the anchor may comprise an inflatable member and may optionally comprise a flow diverter disposed over the distal portion of the elongate member. The flow diverter may comprise a proximal opening, a distal opening, and a lumen extending therebetween, and arranged such that the proximal and distal openings are located outside of the inflatable member. In some variations, the anchor may be a self-expanding anchor. For example, the anchor may comprise a compressible scaffold structure or a compressible cage. Any of the anchors described herein may comprise a shape-memory material.

Also disclosed here are shock wave valvuloplasty methods. One variation of a shock wave valvuloplasty method may comprise advancing a shock wave valvuloplasty device to an aortic valve, where the device may comprise a central tubular member carrying at least two balloon catheters, each balloon catheter including central carrier having a shock wave generator, each balloon catheter further including a balloon affixed to the distal end of the carrier, and a central anchor extending between and beyond the ends of the balloons, advancing the central anchor through the aortic leaflets and into the ventricle, deploying the central anchor within the ventricle to stabilize the position of the balloon catheters, inflating the balloon of at least one of the balloon catheters with a conductive fluid to seat the at least one balloon within the cusp of an aortic leaflet, and activating the shock wave generator to initiate one or more shock waves. In some variations, the shock wave generator of each of the balloon catheters may comprise a pair of electrodes. In this variation, activating the shock wave generator may comprise applying a high voltage across said pair of electrodes such that a plasma arc is created across the electrodes resulting in a shock wave that propagates through the associated balloon. Deploying the central anchor may comprise expanding the anchor from a compressed delivery configuration to an expanded deployed configuration. The central anchor may comprise an anchor balloon or a shape-memory scaffold structure. In some variations, the shock wave generator of each of the balloon catheters may comprise a laser light source, and activating the shock wave generator may comprise generating laser light of sufficient energy to generate a shock wave that propagates through the associated balloon.

Another variation of a shock wave valvuloplasty method may comprise advancing a shock wave valvuloplasty device to an aortic valve, the device comprising an elongated hollow carrier, said carrier including at least one pair of electrodes, a balloon affixed to the distal end of the carrier and a positioning wire, slidably received within the carrier. The at least one pair of electrodes may be located within the balloon and the positioning wire may have a bend formed along a length of the wire that is located within the balloon. The valvuloplasty method may further comprise inflating the balloon with a conductive fluid to seat the at least one balloon within the cusp of an aortic leaflet, adjusting the carrier with respect to the positioning wire, where the bend in the wire varies the angular orientation of the pair of electrodes and thereby adjusts the propagation direction of the shock wave, and applying a high voltage across said pair of electrodes such that a plasma arc is created across the electrodes resulting in a shock wave that propagates at a first direction from a first location through the balloon. Some methods may further comprise moving the carrier with respect to the positioning wire to adjust the angular orientation and location of the pair of electrodes and applying a high voltage across said pair of electrodes such that a plasma arc is created across the electrodes resulting in a shock wave that propagates at a second direction from a second location through the balloon that is different from the first direction and the first location. Any of the valvuloplasty methods described herein may optionally comprise clamping the aortic leaflet between an inflated balloon of at least one of the balloon catheters and the deployed central anchor.

### BRIEF DESCRIPTION OF DRAWINGS

FIG. 1A shows a perspective view of a preferred embodiment of a shock wave valvuloplasty device. FIG. 1B shows a perspective view of a distal portion of the shock wave valvuloplasty device of FIG. 1A. FIG. 1C shows a perspective view of a distal portion of a variation of shock wave valvuloplasty device.

FIG. 2 shows a perspective view of a distal portion of a variation of a balloon, elongated carrier, and electrode assemblies.

FIGS. 3A and 3B show transverse cross-sections of variations of 4-grooved and 6-grooved elongated carriers, respectively. FIG. 3C shows a side view of a 4-grooved elongated carrier.

FIG. 4 shows a close-up view of a portion of the balloon, elongated carrier, and electrode assembly of FIG. 2.

FIG. 5A shows a perspective view of a variation of an electrode assembly. FIG. 5B shows a simplified top view of the electrode assembly of FIG. 5A and a tricuspid aortic valve.

FIG. 6 shows a simplified top view of a variation of an electrode assembly and a cusp of an aortic valve.

FIG. 7 shows a side view of a variation of a positioning wire and electrode assemblies.

FIGS. 8A and 8B show perspective views of a variation of a distal portion of a balloon, an elongated carrier, and electrode assemblies in a first position and a second position, respectively, relative to a positioning wire and an aortic valve cusp.

FIGS. 9A and 9B show side views of variations of a distal portion of a shock wave valvuloplasty device in proximity to a simplified aortic valve. FIG. 9C shows a simplified cross-sectional view of a portion of the shock wave valvuloplasty devices of FIGS. 9A and 9B in relation to an aortic valve.

FIGS. 10A and 10B show photographs of a variation of an anchor in a low-profile and expanded configuration, respectively.

FIG. 11A depicts a perspective view of one variation of a shock wave valvuloplasty device. FIG. 11B is a close-up view of the balloon anchor of the shock wave valvuloplasty device of FIG. 11A. FIG. 11C is a side perspective view of one variation of a flow diverter that may be used with the balloon anchor of FIGS. 11A and 11B. FIG. 11D is a cross-section of the flow diverter of FIG. 11C.

FIG. 12 is an elevational view of one variation of a self-expanding anchor that may be used with a shock wave valvuloplasty device.

### DETAILED DESCRIPTION

Described in detail herein are shock wave valvuloplasty devices and methods that may facilitate treatment of a calcified heart valve through the use of shock waves. Generally, this device may be introduced into a patient's vasculature and advanced to engage a heart valve (e.g., the aortic valve). A distal treatment portion of the device may comprise one or more balloons, each of which may engage a cusp or leaflet of the valve. One or more electrode assemblies within each balloon may generate shock waves, and one or more positioning wires (e.g., nitinol positioning wires) may orient the one or more balloons and/or the direction of the shock waves towards portions of the valve area. A shock wave valvuloplasty device may comprise an anchor that may aid in positioning the distal treatment portion of the shock wave valvuloplasty device at a heart valve and/or augment the shock wave energy delivered to a valve. A proximal portion of the shock wave valvuloplasty device may comprise a proximal control to manipulate features on the distal treatment portion of the shock wave valvuloplasty device. A central tubular member may connect the distal treatment portion of the shock wave valvuloplasty device to the proximal portion of the shock wave valvuloplasty device, and may comprise one or more lumens to contain portions of connectors that may extend between these two portions of the shock wave valvuloplasty device.

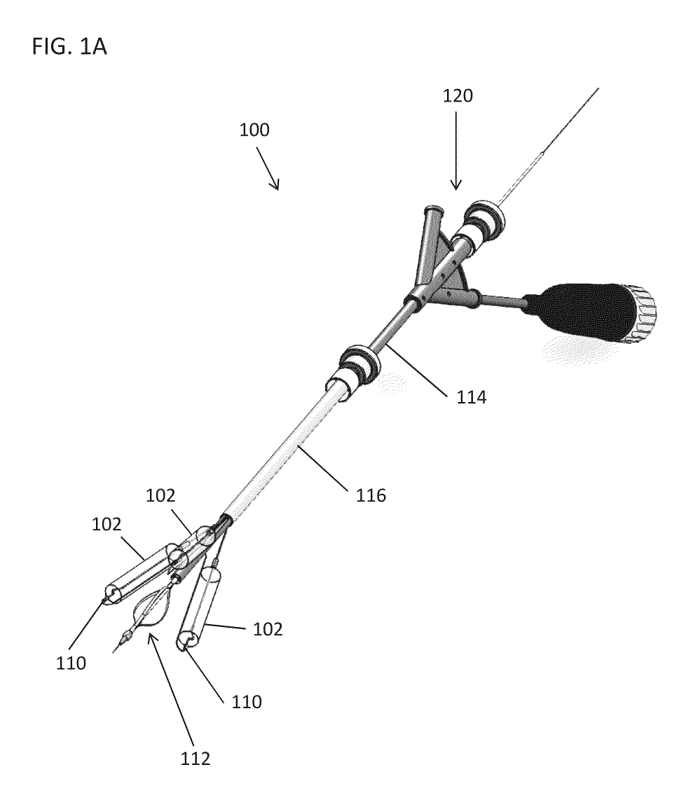

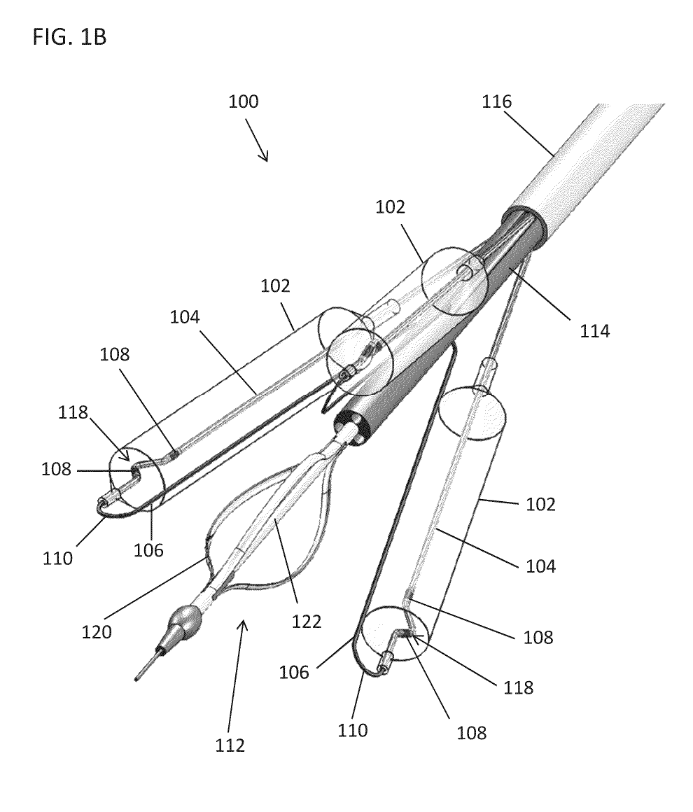

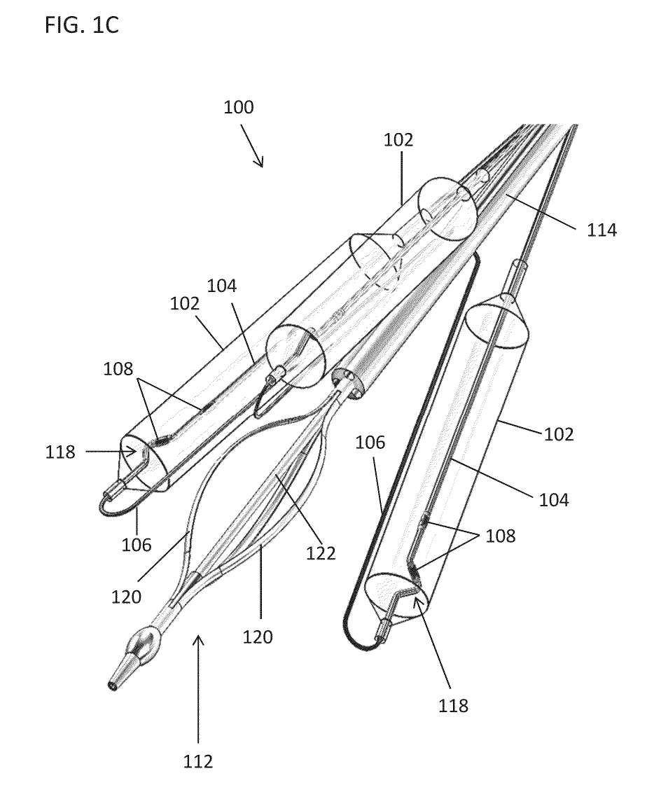

A preferred embodiment of shock wave valvuloplasty device 100 is shown in FIG. 1A, and a magnified view of a distal treatment portion of this shock wave valvuloplasty device is shown in FIG. 1B. A distal treatment portion of the shock wave valvuloplasty device 100 comprises three balloons 102, which may be sized and oriented to each engage one of three cusps of a calcified tricuspid aortic valve. Each balloon 102 sealably encloses a distal portion of an elongated carrier 104, which may be slidably disposed around a portion of a positioning wire 106. One or more electrode assemblies 108 are positioned on each elongated carrier 104, and each electrode assembly may comprise one or more shock wave generators. A balloon may be filled with a liquid, and shock waves generated at an electrode assembly may propagate outward from an elongated carrier through the liquid. Shock waves within a balloon may result in displacement of the balloon wall, which may result in mechanical force being applied to an adjacent calcified valve cusp. This mechanical force may break apart or otherwise disrupt the calcification. While the balloon may be in contact with valve tissue, mechanical force may be transferred to the valve tissue even if there is a gap between the balloon and the valve tissue. Shock waves generated within a balloon may propagate past a balloon wall and travel through blood and soft tissue to transfer mechanical energy to a calcified lesion. While shock waves may be generated using the electrode assemblies described herein, in other variations, shock waves may also be generated using a laser light source. For example, a light-based shock wave generator may comprise a laser light source located outside of the body (i.e., at the proximal end of the catheter) and one or more optical fibers that extend from the laser light source to a distal portion of the elongated carrier within the balloons. The laser light source may generate a light pulse that is transmitted across the optical fiber into the balloon. The light pulse may have a sufficient energy level or power density to generate a shock wave that travels through the fluid and past the balloon wall to transfer mechanical energy to a calcified lesion.

The energy of a shock wave diminishes as it propagates away from its source. Therefore, the amount of mechanical energy transferred from a shock wave to calcified tissue may be increased by moving a shock wave generator closer to the calcified tissue. As the shock wave generators may be fixed to a position on an elongated carrier within a balloon, movement of a balloon closer to calcified tissue may increase the mechanical energy transferred from shock waves to the calcified tissue. A shock wave valvuloplasty device may therefore comprise one or more elements to bring one or more balloons close to calcified valve tissue. Advancement of the shock wave valvuloplasty device within a patient's vasculature may position a balloon in proximity to a valve. As seen in FIG. 1B, this process may be facilitated by a distal bend 110 in each positioning wire 106 that may engage a valve cusp. A user may feel resistance to advancement when a distal bend 110 of a positioning wire 106 contacts a valve cusp, which may indicate that the shock wave valvuloplasty device 100 has been advanced to a desired location (e.g., the aorta side of the aortic valve). In some variations, the shock wave valvuloplasty device 100 may comprise an anchor 112 that may cross the aortic valve and enter the left ventricle. The anchor 112 may be expanded in the left ventricle, which may prevent the anchor from moving proximally out of the ventricle, and may maintain the shock wave valvuloplasty device 100 in a desired position at the aortic valve.

The anchor 112 may comprise one or more arms 120 that may facilitate alignment of each balloon 102 with a valve cusp. Expansion of the anchor 112 in the left ventricle results in the arms 120 bowing out from an outer shaft 122 of the anchor. In the anchor orientation shown in FIG. 1B, each arm 120 is circumferentially aligned with a balloon 102, such that each valve cusp may be positioned between a balloon and an anchor arm when the anchor is expanded in the left ventricle. This may help retain each balloon 120 in proximity to a valve cusp. In the anchor orientation shown in FIG. 1C, the arms 120 are rotated around the outer shaft 122 of the anchor 112 about 60 degrees compared to the orientation shown in FIG. 1B. In this configuration, each arm 120 is circumferentially offset about 60 degrees compared to each balloon 102 (i.e. circumferentially halfway between each balloon). In this configuration, the anchor 112 may be advanced through the aortic valve with the arms 120 bowed out from the outer shaft 122 by aligning each arm with a space between two adjacent aortic valve cusps (an intercusp space). Aligning each arm 120 with each intercusp space of an aortic valve may approximately align each balloon 102 with the center of each valve cusp.

The radial position of a balloon 102 relative to the longitudinal axis of the central tubular member 114 may be controlled, which may bring a balloon closer to a desired area of a valve cusp. As shown in FIG. 1B, the shock wave valvuloplasty device may comprise an overtube 116, which may be slidably disposed around a portion of the central tubular member 114. The overtube 116 may be advanced distally along the central tubular member 114 and alter the position of a positioning wire 106, such that a balloon 102 disposed around a portion of this positioning wire may be drawn closer to the longitudinal axis of the central tubular member. A balloon 102 may be further positioned in relation to a valve cusp by distal advancement or proximal retraction along a positioning wire 106.

Radial and longitudinal displacement of a balloon relative to a valve cusp may accordingly radially and longitudinally displace the one or more electrode assemblies that are located on the elongated carrier in the balloon. Additionally or alternatively, the angular orientation of an elongated carrier relative to a valve cusp may be altered, which may alter the angular orientation of one or more electrode assemblies positioned on this elongated carrier. As shown in FIGS. 1B and 1C, the positioning wire 106 may comprise one or more kinks 118, over which an elongated carrier 104 may slide. Positioning an electrode assembly 108 over a kink may alter the angular orientation of an electrode assembly relative to a valve cusp. Shock waves may be generated from an electrode assembly and may propagate from the electrode assembly in a specific direction based on the orientation of the electrode assembly. As described, the shock wave valvuloplasty device may comprise features that may allow a user to alter the longitudinal, radial, and angular orientation of one or more electrode assemblies, which may facilitate the direction of shock waves at a specific portion or portions of calcified valve. This may be advantageous in order to treat the entire valve by sweeping the valve area with shock waves, or may allow a user to direct the shock waves at specific areas (e.g., areas of greatest calcification). The various components of a shock wave valvuloplasty device will be described in more detail herein.

A shock wave valvuloplasty device may comprise one or more balloons (e.g., one, two, three) that may each be positioned adjacent to the concave side of a heart valve cusp. In some variations, the shock wave valvuloplasty device may comprise one balloon, and this balloon may deliver shock waves to a valve cusp and then be moved to a different cusp to deliver shock waves to that cusp. In some variations, it may be advantageous for a shock wave valvuloplasty device to comprise more than one balloon, which may facilitate self-alignment of the more than one balloon adjacent to more than one valve cusp. In some variations, it may be desirable for a shock wave valvuloplasty device to comprise two balloons, such as for use in individuals with a bicuspid aortic valve. In some variations it may advantageous for a shock wave valvuloplasty device to comprise three balloons, such as for use in individuals with a tricuspid aortic valve. Positioning each of the three balloons adjacent to each of the three cusps of an aortic valve may facilitate delivering shock waves to each cusp without needing to move a balloon from one cusp to another, which may decrease the time of the valvuloplasty procedure. In some variations, more than one balloon may deliver shock waves to more than one cusp simultaneously, which may further decrease the time of the valvuloplasty procedure.

A balloon may be shaped and sized to conform to the shape of a valve cusp (e.g., the shape of a concave side of an aortic valve cusp) when filled with a fluid. This may increase the mechanical force transferred to a calcified valve by a shock wave generated in the balloon and/or may facilitate a balloon automatically positioning itself in a cusp. A balloon may be spherical or it may have any other suitable shape (e.g., tetrahedron with rounded and/or sharp corners or edges, square-circle-triangle block) that may facilitate positioning in a valve cusp. The balloon material may be compliant, which may facilitate conforming to the shape of a valve, or it may be non-compliant and molded to the shape of a valve anatomy. For example, a balloon may comprise one or more indentations that may decrease the risk of an inflated balloon occluding an opening to a coronary artery while the balloon is adjacent a valve cusp.

A balloon may be sized to fit within the concave portion of a valve cusp when inflated with a fluid. For example, in some variations, the balloon may have a transverse diameter between 4 mm and 8 mm. The size of the balloon may be such that at least a portion of the pre-procedure movement of a calcified valve cusp may be maintained while the balloon is adjacent to the valve cusp. This may decrease any negative effect a shock wave valvuloplasty device may have on an aortic valve pressure gradient (the pressure difference between the ventricular side and aortic side of the aortic valve) and on cardiac output during the valvuloplasty procedure. Patients with aortic valves that are calcified to a degree that they experience aortic stenosis may be especially sensitive to decreases in cardiac output and/or to increases in left ventricular pressure. In variations of the shock wave valvuloplasty device that comprise one or more balloons, the one or more balloons may be inflated and/or deflated with fluid simultaneously or separately. In some variations, it may be advantageous for only one balloon to be inflated at a time during at least a portion of the valvuloplasty procedure. This may further decrease any negative effects the shock wave valvuloplasty device may have on the aortic valve pressure gradient and/or on cardiac output while the shock wave valvuloplasty device is adjacent to the aortic valve. Each balloon may be connected to a fluid channel that may extend through a portion of the central tubular member to a portion of the proximal control. The proximal control may comprise one or more ports that may be used to introduce and/or withdraw fluid to inflate and/or deflate, respectively, one or more balloons simultaneously or separately. The fluid may comprise any suitable liquid (e.g., saline, saline/contrast mixture). In some variations, a balloon may comprise one or more radiopaque materials. In some variations, a balloon may comprise more than one material, which may be layered. In some variations, the balloon material may be homogeneous. The one or more materials of a balloon may have a sufficient strength and/or compliance to tolerate rapid fluctuations in volume during shock wave generation. The one or more materials of a balloon may be heat resistant to reduce the risk of damage that may occur to the balloon as a result of heat emitted by an electrode assembly during shock wave generation.

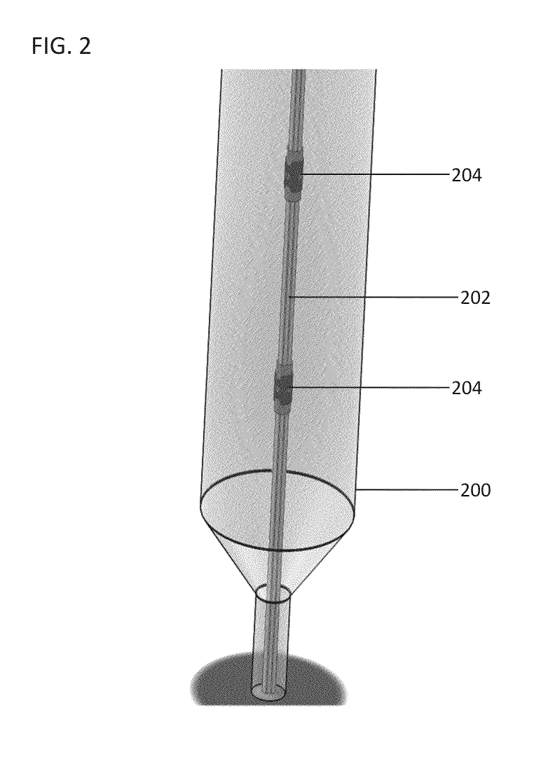

Each balloon of a shock wave valvuloplasty device may sealably enclose a distal portion of an elongated carrier (e.g., a catheter comprising a lumen). FIG. 2 shows a portion of a balloon 200 and elongated carrier 202. For clarity, a positioning wire is not shown in this figure. Two electrode assemblies 204 are positioned on the outer surface of the elongated carrier 202, although it should be appreciated that any suitable number (1, 2, 3, 4, 5, 6, 7, 8) of electrode assemblies may be located on an elongated carrier. An elongated carrier may comprise an elongated carrier lumen that may slidably receive a portion of a positioning wire, such that the elongated carrier may be advanced distally and retracted proximally over the positioning wire. The balloon and electrode assemblies are fixed to the elongate carrier, and any movement of the elongated carrier over the positioning wire also moves the balloon and electrode assemblies. As will be discussed in more detail, a positioning wire may comprise one or more curves or kinks, and at least the portion of an elongated carrier that may be advanced and/or retracted over the one or more curves or kinks may be flexible.

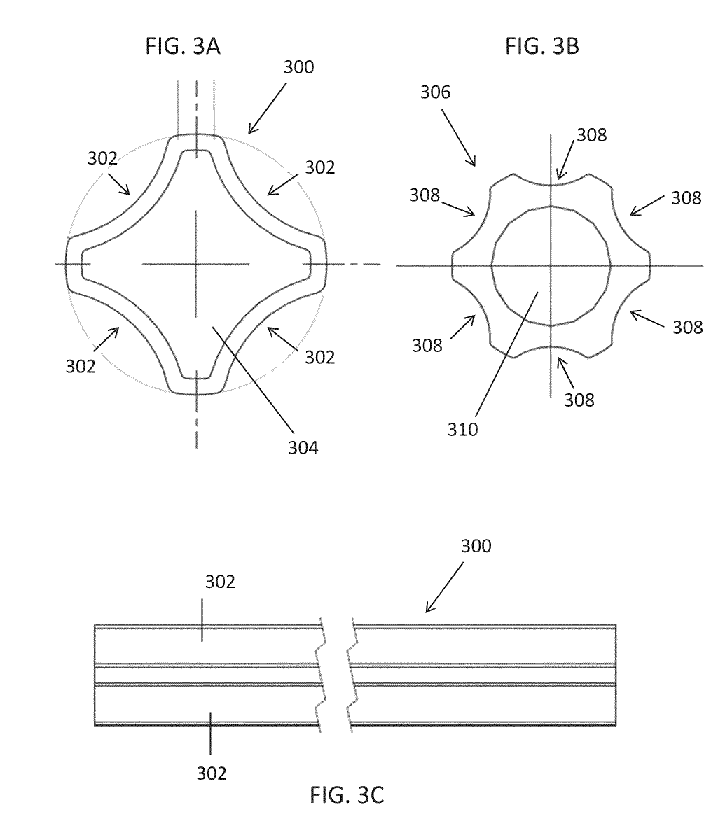

An elongated carrier may be configured to accommodate one or more portions of an electrode assembly and/or conductors that may connect an electrode assembly to a power source. For example, one or more wires may be positioned in a wall of the elongated carrier. In some variations, an elongated carrier may comprise one or more longitudinal grooves that may accommodate portions of one or more electrode assemblies and/or one or more wires that connect to an electrode assembly. For example, FIGS. 3A-3C depict elongated carriers comprising longitudinal grooves. FIG. 3A shows a cross-sectional view of an elongated carrier 300 that comprises four longitudinal grooves 302 circumferentially spaced around an elongated carrier lumen 304. FIG. 3B shows a cross-sectional view of an elongated carrier 306 that comprises six longitudinal grooves 308 circumferentially spaced around an elongated carrier lumen 310. FIG. 3C shows a side view of the elongated carrier 300 of FIG. 3A, where two longitudinal grooves 302 are seen extending parallel to a longitudinal axis of the elongated carrier 300. While elongated carriers are shown comprising 4 and 6 grooves, it should be appreciated that an elongated carrier may not comprise grooves or may comprise any suitable number of grooves (e.g., 1, 2, 3, 4, 5, 6, 7, 8, 9, 10). In some variations, it may be advantageous for an elongated carrier to comprise grooves to accommodate portions of one or more wires that connect to electrode assemblies. A configuration with wires in grooves may allow the elongated carrier and wires to be more flexible than they may be with the wires positioned within the walls of the elongated carrier. This may facilitate movement of an elongated carrier over curves or kinks in a positioning wire.

It should be appreciated that the cross-sectional shape of the elongated carrier lumen may have any suitable shape (circle, oval, rectangular). In some variations, the shape of the elongated carrier lumen may be such that it accommodates the shape of a positioning wire that may be partially disposed within the lumen. For example, in some variations the positioning wire may be rectangular and the elongated carrier lumen may be rectangular. This configuration may prevent the elongated carrier from rotating around the positioning wire. In variations where the elongated carrier is prevented from rotating around the positioning wire, each groove, and any element within a groove (e.g., an electrode) may be in a fixed position relative to the positioning wire. This may facilitate the propagation of shock waves in specific, predictable directions relative to the positioning wire.

One or more electrode assemblies may be positioned on an outer surface of each elongated carrier to produce shock waves. An electrode assembly may be low-profile and layered in a configuration that may allow for shock waves to be directed outward from the side of an elongated carrier. Electrodes of this nature are described in detail in U.S. Pat. Publ. No. 2014/0052147, which is hereby incorporated by reference in its entirety. Generally, a low-profile, layered electrode assembly may comprise an inner electrode, an outer electrode, and an insulating layer between them. The insulating layer may have a first opening and the outer electrode may have a second opening that is coaxially aligned with the first opening, such that a portion of the inner electrode is exposed by the openings. The inner and outer electrode may be connected to a high voltage source by wires, and configured such that the inner electrode is a positive terminal and the outer electrode is a negative terminal (or vice-versa). By applying a high voltage pulse between the inner and outer electrodes, a gas bubble may be generated at the surface of the electrodes and a plasma arc of electric current may traverse the bubble and create rapid expansion and contraction of the bubble. This may create a mechanical shock wave in a fluid-filled balloon that may be transferred to a valve cusp in order to break apart or otherwise disrupt calcified plaques.

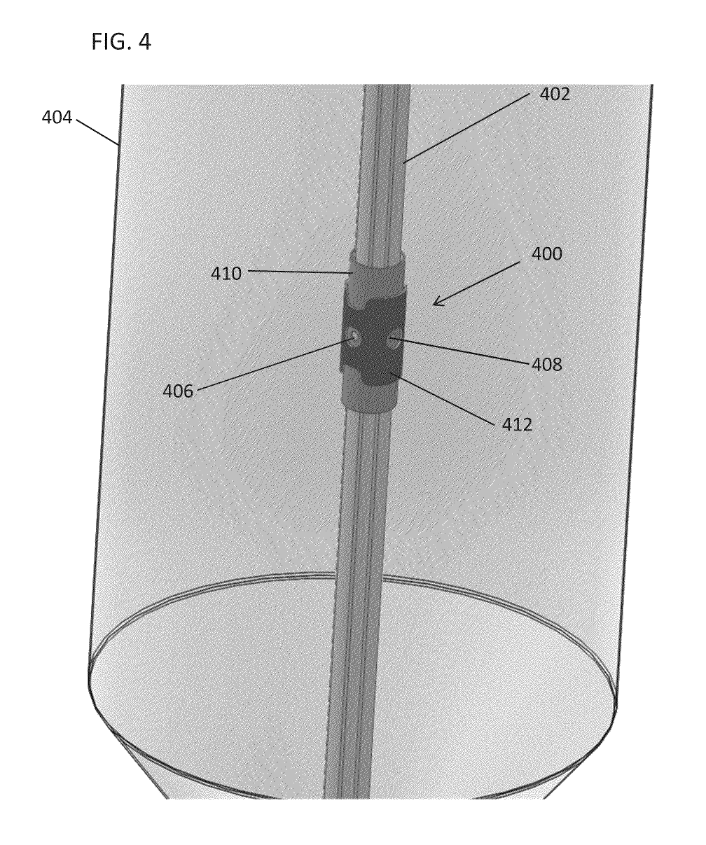

FIG. 4 depicts a variation of electrode assembly 400 positioned on an outer surface of an elongated carrier 402 within a balloon 404. The variation shown comprises a first inner electrode 406 and a second inner electrode 408. Only a portion of these inner electrodes 406 and 408 are visible through openings in an insulating layer 410 and outer electrode 412. While two inner electrodes are shown in this variation, it should be appreciated that an electrode assembly may comprise any suitable number of electrodes (e.g. one, two, three, four, five, six). An inner electrode may be substantially co-planar with the outer surface of the elongated carrier and comprise any suitable shape. In some variations, at least a portion of an inner electrode may be positioned within a groove of the elongated carrier. In some variations, an inner electrode may be a hypotube that wraps around the circumference of the elongated carrier. As shown in FIG. 4, the insulating layer 410 may be a sheet or sheath that wraps at least partially around the circumference of the elongated carrier 402 and overlaps the one or more inner electrodes 406 and 408. The insulating layer may overlap an inner electrode such that the inner electrode is electrically isolated from the environment external to the elongated carrier, except for the portion of the inner electrode exposed by the opening in the insulating layer. In some variations, an insulating layer may be an insulating coating directly applied over an inner electrode surface, while leaving a conductive portion of the inner electrode uncoated. The outer electrode may be a ring, sheet, or sheath that may be circumferentially wrapped over the insulating layer. In some variations, an outer electrode may be a radiopaque marker band.

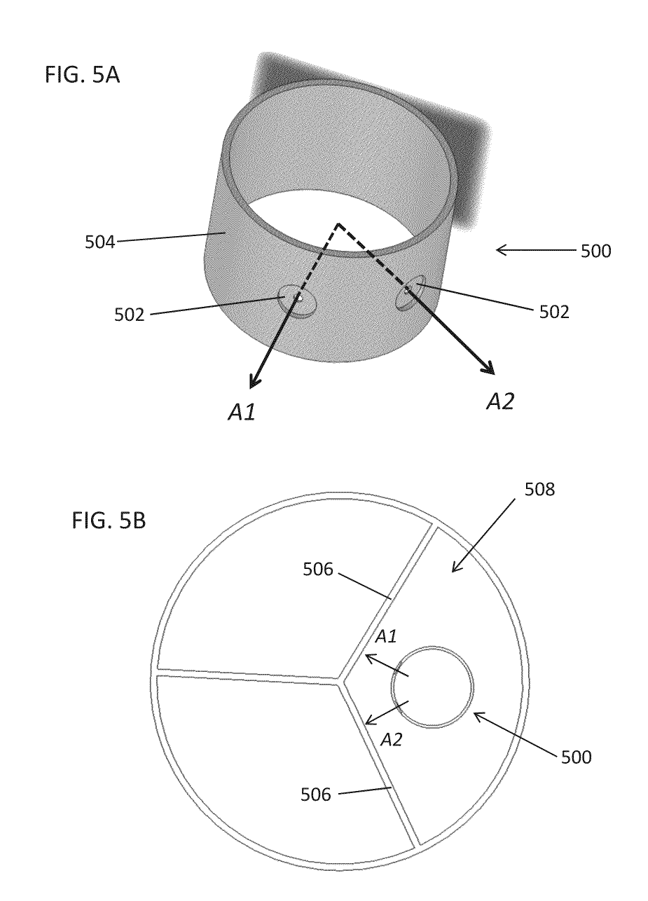

The outer electrode may be positioned such that the opening in the outer sheath may be coaxially aligned with the opening in the insulating layer. In the variation shown in FIG. 4, the openings are concentric circles, but the openings may be any suitable shape (oval, square, rectangle). The position of the openings may allow for the shock waves that propagate outward from the sides of the elongated carrier to be directed. For example, FIGS. 5A and 5B show a variation of electrode assembly 500 comprising a first inner electrode (not pictured) and second inner electrode (not pictured). Associated with each inner electrode are aligned openings of an insulating layer 502 and outer electrode 504. The direction of shock waves generated by the current between the first inner electrode and outer electrode is indicated by arrow A1 and the direction of shock waves generated by the current between the second inner electrode and outer electrode is indicated by arrow A2. The position of electrodes and openings may direct shock waves in any relative direction to one another. For example, in FIGS. 5A and 5B, the shock waves are circumferentially displaced by about 60 degrees. The electrodes and openings may be positioned in order to direct shock waves at specific areas of valve anatomy. For example, FIG. 5B depicts a top view of the electrode assembly 500 of FIG. 5A as it may appear positioned relative to a tricuspid aortic valve. In this example, the direction of shock waves, as indicated by arrows A1 and A2 may be directed towards the inner, free margins 506 of a valve cusp 508.

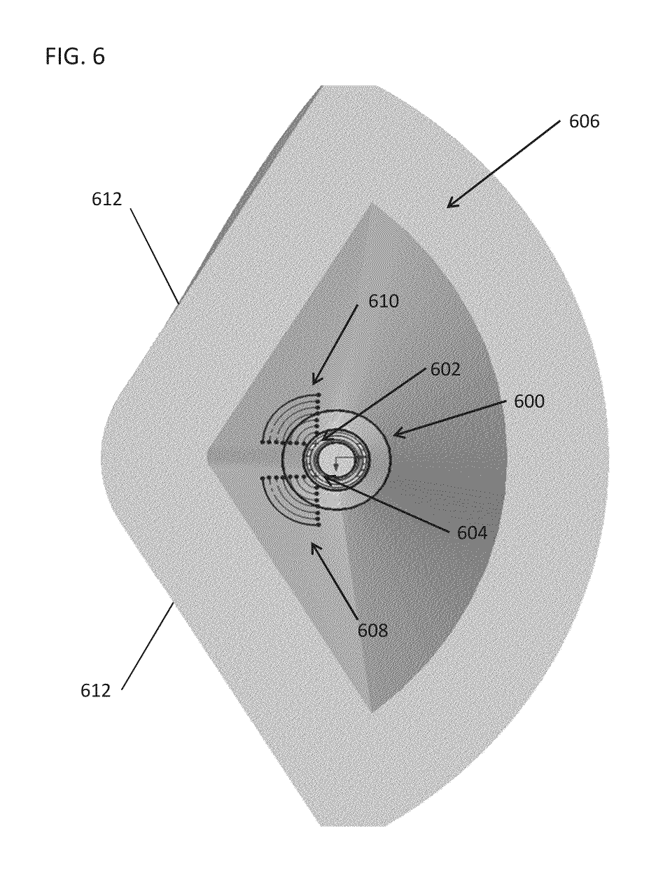

FIG. 6 similarly depicts a top view of an electrode assembly 600 comprising two shock wave generators (e.g., layered inner and outer electrodes) 602 and 604 as they may appear in relation to a cusp 606 of a tricuspid aortic valve. The shock wave generators are circumferentially displaced about 60 degrees from one another and may generate shock waves that propagate away from the electrode assembly in the pattern depicted by arcuate lines 608 and 610. As shown, the shock waves may be directed towards specific areas of a valve anatomy, in this case towards the inner, free margins 612 of a cusp.

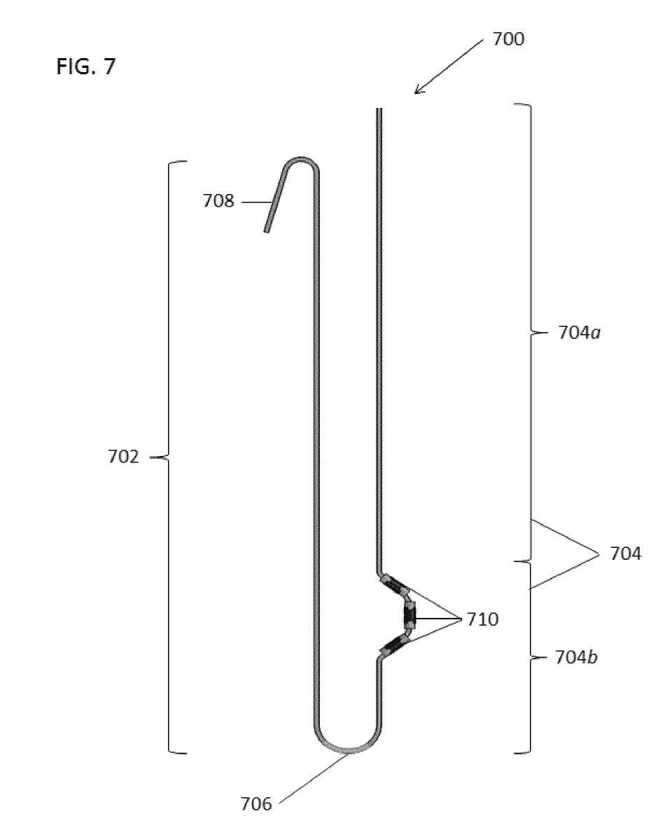

Advancement or retraction of an elongated carrier and balloon over a positioning wire may further affect the direction of shock wave propagation. A shock wave valvuloplasty device may comprise a positioning wire for each elongated carrier and balloon to move over. FIG. 7 shows a positioning wire 700 in isolation, similar to the positioning wires 106 of FIGS. 1B and 1C, but with a slightly different shape. A positioning wire may comprise a support region 702 and a positioning region 704 separated by a U-shaped distal bend 706. The support region may be secured to the central tubular member (not pictured) in any suitable way in order to stabilize the positioning wire. As shown, the support region comprises a hook 708 to attach to the central tubular member. A balloon (not shown), elongated carrier (not shown), and one or more electrode assemblies 710 may be slidably disposed around a portion of the positioning region 704 of the positioning wire 700, and may not be advanced past the distal bend 706. The positioning region 704 further comprises a straight, proximal region 704a and a curved or kinked, distal region 704b. An elongated carrier may be positioned on the positioning region such that the balloon and one or more electrode assemblies on the elongated carrier are proximal to the kinked positioning region. The elongate carrier may be advanced distally, which may position at least a portion of the kinked positioning region of the positioning wire within the elongated carrier lumen. The elongated carrier may be flexible in order to conform to and slide over the kinked portion of the positioning wire. An electrode assembly on the elongated carrier may similarly be positioned on the straight region of the positioning wire or advanced over the kinked region. FIG. 7 shows the orientation of three electrode assemblies 710 positioned over the kinked region 704b of the positioning wire 700. The balloon and elongated carrier have been removed from this figure for clarity. The size of the electrode assemblies 710 and angles of the kinked region 704b may be such that the electrode assemblies may slide over the kinked region.

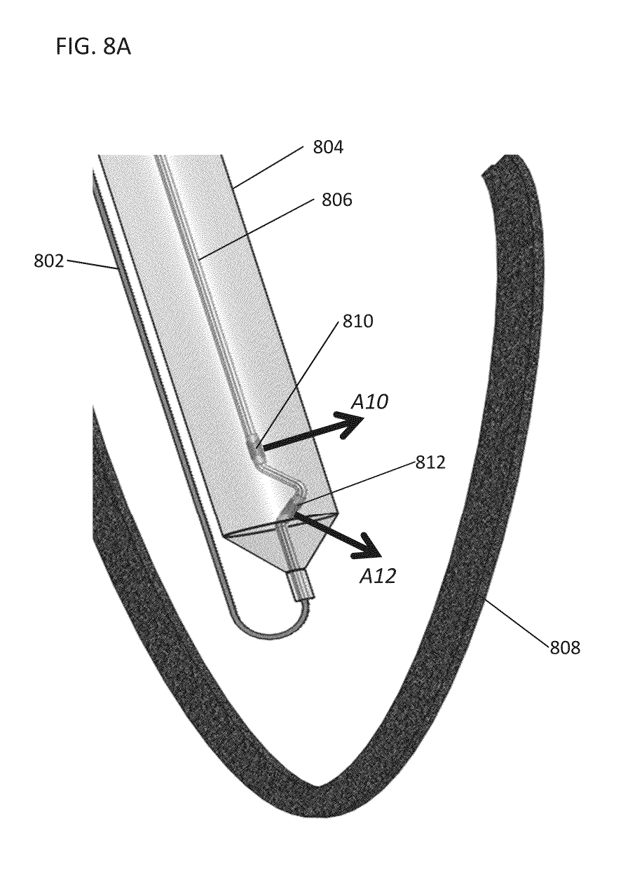

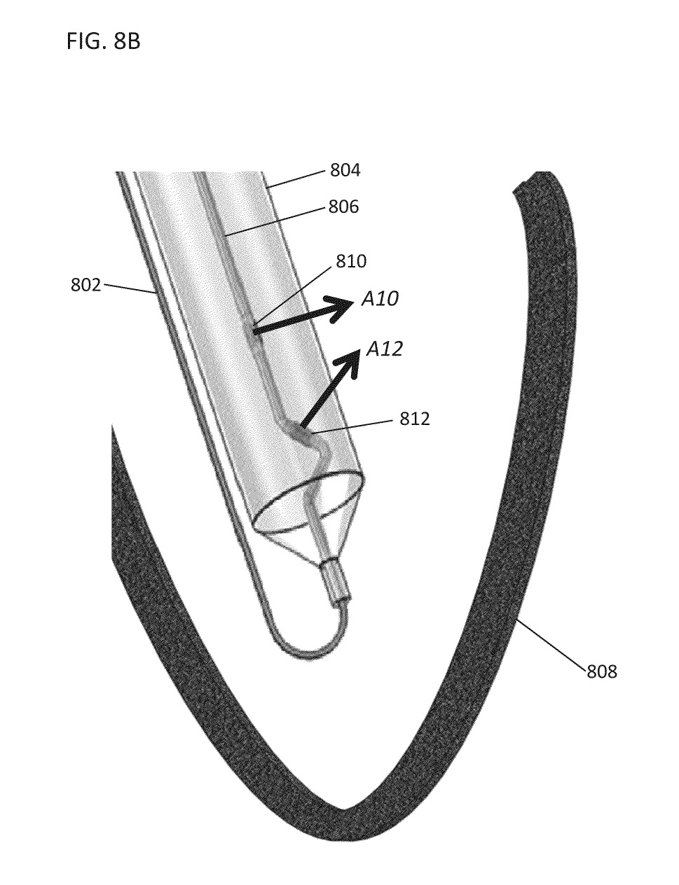

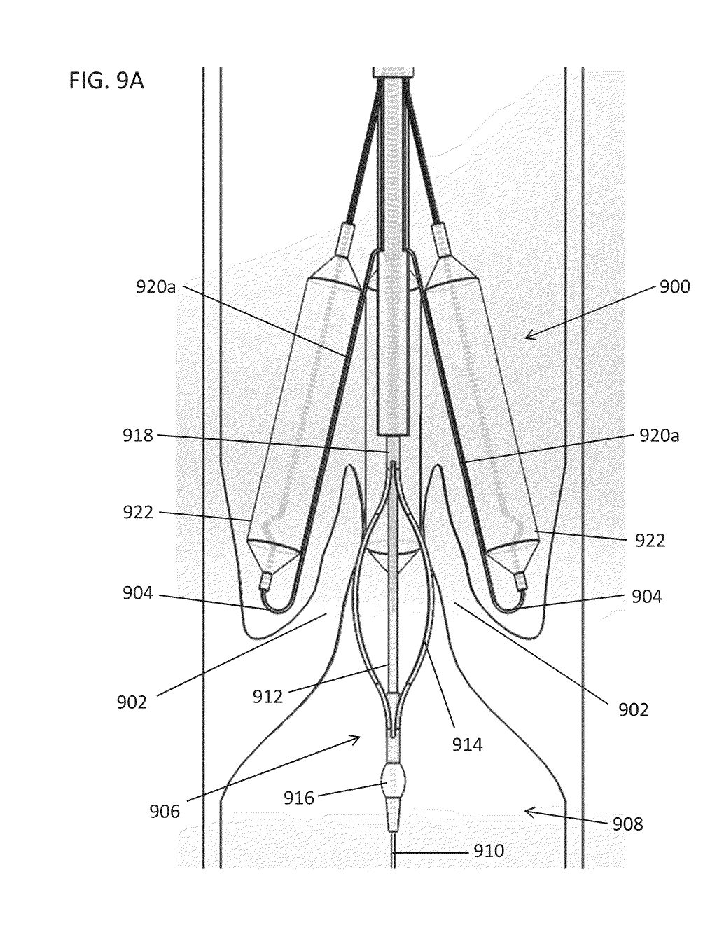

The orientation of the positioning wire over which an electrode assembly is positioned may determine the primary direction of the shock wave propagation. This may allow shock waves to be directed at specific portions of a valve anatomy. FIGS. 8A and 8B illustrate how the orientation of a kinked region may determine the primary direction of shock wave propagation. FIGS. 8A and 8B show a distal portion of a positioning wire 802, balloon 804, and elongated carrier 806 in proximity to a valve cusp 808. In FIG. 8A, a first electrode assembly 810 of the elongated carrier 806 is located on the straight region of the positioning wire 802 and a second electrode assembly 812 is located on a kinked region of the positioning wire 802. The shock waves generated by the first electrode assembly 810 may propagate in the plane of arrow A10, which is perpendicular to the portion of positioning wire that the first electrode assembly is positioned over. The portion of the kinked region that the second electrode assembly 812 is positioned over is angled relative to the straight region that the first electrode 810 is positioned over. Accordingly, the direction of shock wave propagation generated by the second electrode assembly (in the plane of arrow A12) is angled inferiorly relative to the direction of propagation from the first electrode assembly. The portion of the valve cusp targeted by shock waves from the second electrode assembly may be more inferior than the portion of the valve cusp targeted by shock waves from the first electrode assembly.

Advancement or retraction of electrode assemblies between different positions on the straight and/or kinked regions of the positioning wire may allow for shock waves to be delivered to multiple areas of a valve anatomy. This may facilitate targeting of specific areas (e.g., the aortic wall, the free, inner margins of each cusp) or may facilitate sweeping the valve area in order to treat a whole region of valve anatomy. For example, the balloon 804, elongated carrier 806, and electrode assemblies 810 and 812 shown in FIG. 8B have been retracted proximally over the positioning wire 802 relative to their positions in FIG. 8A. The first electrode assembly 810 is on a more proximal straight portion of the positioning wire 802 in FIG. 8B compared its position in FIG. 8A, which may direct shock waves in the plane of arrow A10 at a more superior portion of the valve cusp 808. The second electrode assembly 812 has been retracted to a more proximal position on the kinked region of the positioning wire in FIG. 8B, as compared to FIG. 8A. The angle of the kinked portion over which the second electrode assembly is positioned is different in FIG. 8B than it is in FIG. 8A. Shock waves produced by the second electrode assembly 812 may propagate in the plane of arrow A12, which is directed superiorly in FIG. 8B, as opposed to inferiorly in FIG. 8A.

A kinked region of a positioning wire may comprise any suitable number of kinks and/or curves with any suitable angles or radii of curvature, such that shock waves may be directed at desired portions of valve anatomy. A positioning wire may be pre-formed with one or more kinks and/or curves in any suitable manner. In some variations, a positioning wire may comprise a shape-memory alloy, such as nitinol. This may be advantageous as one or more curved portions of a positioning wire may be constrained and straightened during portions of the valvuloplasty procedure (e.g., introduction and advancement of the shock wave valvuloplasty device in vasculature) where a smaller device profile may be desirable. In some variations, the positioning wire may be rectangular and flat, such as a ribbon wire. As mentioned, a portion of this wire may be slidably disposed within an elongated carrier lumen. In some variations, the cross-sectional shape of the elongated carrier lumen may be rectangular to match the shape of the positioning wire. This configuration may prevent rotation of the elongated carrier around the positioning wire, which may be advantageous by maintaining shock wave generators in specific, predetermined locations relative to the positioning wire. This may allow a user to anticipate where shock waves will be directed and manipulate the shock wave valvuloplasty device accordingly.

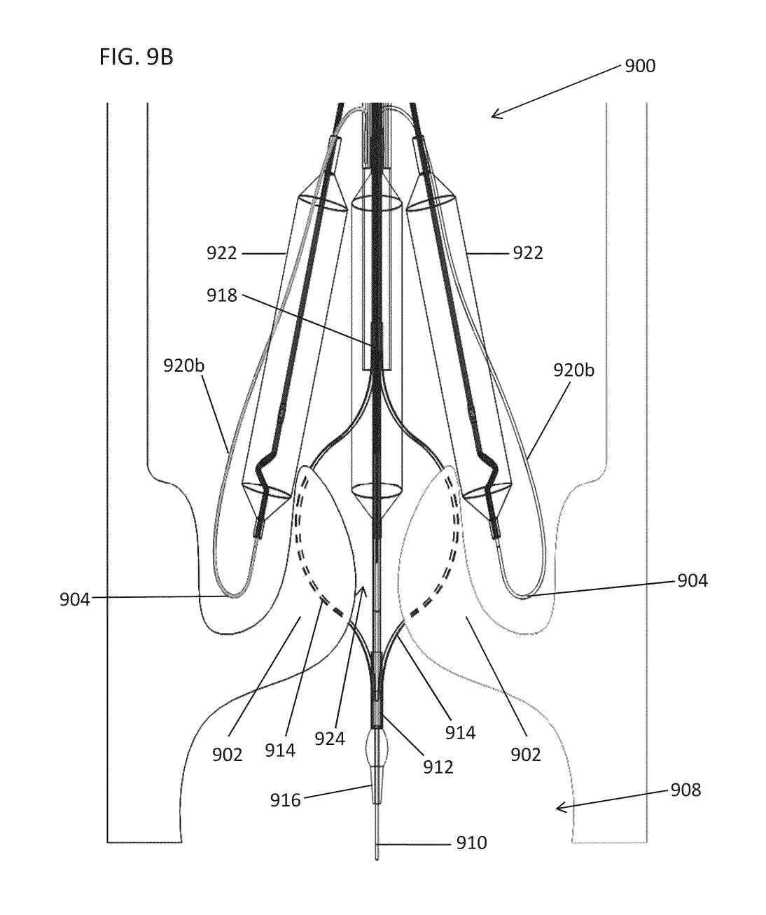

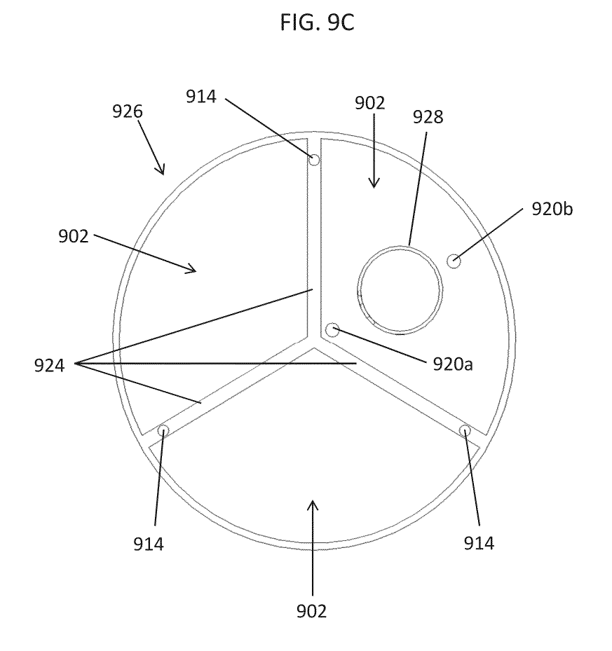

In some variations, the support region of the positioning wire may be configured to avoid interference with shock wave propagation. For example, FIGS. 9A and 9B show side views of distal portions of shock wave valvuloplasty devices 900 with different orientations of positioning wires. In FIG. 9A, the support regions 920a of the positioning wires are positioned between the balloons 922 and inner portions of the valve cusps 902. In other variations, as shown in FIG. 9B, the support regions 920b of the positioning wires are positioned between the balloons 922 and outer portions of the valve cusps 902. These two alternate positioning wire orientations are also seen in FIG. 9C. FIG. 9C shows a transverse cross-sectional view of an electrode assembly 928 and the two alternate orientations of the positioning wire support regions 920a and 920b. The location of the support region 920a, oriented as shown in FIG. 9A, is seen in FIG. 9C between the electrode assembly 928 and an inner portion of a valve cusp 902. The location of the support region 920b, oriented as shown in FIG. 9B, is seen in FIG. 9C between the electrode assembly 928 and an outer portion of a valve cusp 902. The orientation of support region 920b may be advantageous by decreasing interference from the positioning wire to shock wave propagation from electrode assemblies to inner portions of the valve cusps.

A positioning wire and overlying balloon may be radially displaced relative to a valve by advancement and retraction of an overtube. As shown in FIGS. 1A and 1B, an overtube 116 may be slidably disposed around a portion of the central tubular member 114 and advanced distally and/or retracted proximally. When the overtube 116 is advanced distally, it may be advanced over a portion of the positioning wire 106, which is deflected at an angle relative to the central tubular member 114. Advancing the overtube 116 over the positioning wire 106 may reduce the angle of deflection and compress the positioning wire closer to the longitudinal axis of the central tubular member 114. As a balloon, elongated carrier, and one or more electrode assemblies are disposed over a portion of the positioning wire, advancing the overtube may also draw these structures closer to the longitudinal axis of the central tubular member. This may move a balloon wall into closer proximity to inner portions of a valve cusp to more efficiently transfer mechanical energy from shock waves to these portions of the valve. Similarly, an overtube may be withdrawn proximally to increase the angle a positioning wire is deflected from a central tubular member, which may bring a balloon into closer proximity to outer portions of a valve cusp.

A shock wave valvuloplasty device may comprise one or more features that may facilitate self-alignment of one or more balloons with a portion of a valve (e.g, the concave portion of an aortic valve cusp). As was described in more detail, the shape and size of a balloon may allow for a balloon to fit within and/or conform to a portion of an aortic valve cusp. A U-shaped distal bend of a positioning wire may also facilitate alignment of a balloon in a valve cusp. For example, FIGS. 9A and 9B show side views of distal treatment portions of shock wave valvuloplasty devices 900 in proximity to aortic valve cusps 902. In each figure, two distal bends 904 of positioning wires are seen in the concave portions of two valve cusps 902. While the position of a shock wave valvuloplasty device within vasculature may be confirmed with imaging (e.g., fluoroscopy, ultrasound), a distal bend may provide tactile feedback to a user when a distal bend contacts a valve cusp, which may indicate the position of the device. For example, when a shock wave valvuloplasty device is advanced to an aortic valve, a distal bend may contact a valve cusp and resist further advancement of the device. This resistance may indicate to a user that the device may be in a desired position to treat the valve. In variations of a shock wave valvuloplasty device comprising more than one positioning wire and distal bend (e.g, two positioning wires for a bicuspid aortic valve, three positioning wires for a tricuspid aortic valve), the spacing of the positioning wires relative to one another may further facilitate advancement of the device to a desired location. For example, the relative spacing of the distal bends may be similar to the relative spacing of the centers of valve cusps, such that each distal bend may be aligned with a center of a valve cusp.

In some variations, a shock wave valvuloplasty device may comprise a central anchor that may cross the aortic valve and enter the left ventricle to facilitate alignment of the device and/or reflection of shock waves. The anchor may be expandable between a low-profile configuration and an expanded configuration. The low-profile configuration may be desirable during advancement of the shock wave valvuloplasty device through vasculature. During advancement, a distal tip of the anchor may cross the aortic valve and enter the left ventricle. Expansion of an anchor within a left ventricle may prevent the anchor from exiting through the aortic valve and may hold the balloons and positioning wires in a desirable position for directing shock waves to the valve cusps. In some variations, the anchor may comprise one or more arms (e.g., 2 arms for use with a bicuspid valve, 3 arms for use with a tricuspid valve) that may extend from a central axis of the anchor. The arms may bow out from the central axis of the anchor more in the expanded configuration than in the low-profile configuration. In other variations, the anchor may comprise one or more wire structures (e.g., wire loops or lobes) that have a collapsed configuration and an expanded configuration. In still other variations, the anchor may comprise one or more balloons having a collapsed configuration and an expanded configuration. One or more of the wire structures or balloons in the expanded configuration may be configured to extend through and/or be seated within or below the valve orifice. In addition to facilitating alignment of the device with respect the valve, the anchor may optionally apply a radial force onto the valve leaflets to help improve the contact between the valve leaflets and the balloons that enclose the shock wave electrodes.

In some variations, as shown in FIG. 9A, each arm 914 may be circumferentially aligned with a balloon 922. In this orientation, a balloon 922 may be positioned adjacent to a concave portion of a valve cusp 902, and an expanded anchor arm 914 may be positioned directly inferior to the balloon, adjacent to a convex portion of the valve cusp. Sandwiching each valve cusp 902 between a balloon 922 and an expanded anchor arm 914 may retain each balloon in proximity to a valve cusp for efficient treatment. In some variations, as shown in FIG. 9B, each anchor arm 914 may be circumferentially oriented halfway between each balloon 922. In this orientation, each anchor arm 914 may be aligned with an intercusp space 924 while each balloon 922 is aligned with the center of a valve cusp 902. This orientation may facilitate self-alignment of each balloon 922 in a valve cusp 902 as the anchor is advanced through the aortic valve. If the anchor is advanced with the arms 914 bowed out from the central axis of the anchor, the anchor may not cross the aortic valve unless the arms align with the intercusp spaces 924. The orientation of anchor arms 914 shown in FIG. 9B is seen in cross-section in FIG. 9C. FIG. 9C shows a transverse cross-section of a distal portion of a valvuloplasty device in relation to an aortic valve 926. Three anchor arms 914 are positioned within three intercusp spaces 924 between three valve cusps 902. This orientation may allow an anchor to be advanced past the aortic valve into the left ventricle while also aligning each balloon (not shown) and electrode assembly 928 with the center of each valve cusp 902.

Additionally, the distal anchor may enhance breaking up or softening calcified tissue by reflecting inferiorly directed shock waves. For example, an electrode assembly may generate shock waves that propagate inferiorly to a concave portion of a valve cusp and deflect the valve cusp inferiorly towards the left ventricle. A portion of the anchor in the left ventricle may contact the convex portion of the valve cusp, and resist inferior deflection of the valve cusp. This may result in the valve cusp being compressed between a shock wave and a portion of the anchor, which may enhance the breakup of calcified valve tissue. The compression of a valve cusp between shock waves and an anchor arm may be facilitated by anchor arms circumferentially aligned with each balloon, as shown in FIG. 9A. In some variations, a shock wave valvuloplasty device may comprise an anchor that may be rotated between two or more positions. For example, in a first position, each anchor arm may be circumferentially halfway between each balloon. This may facilitate self-alignment of each balloon with the center of a valve cusp when the anchor arms are advanced through intercusp spaces. Once advanced into the left ventricle, the anchor may then be rotated about 60 degrees relative to the position of the balloons, which may circumferentially align each arm with each balloon. This orientation may facilitate the reflection of shock waves by anchor arms during treatment.

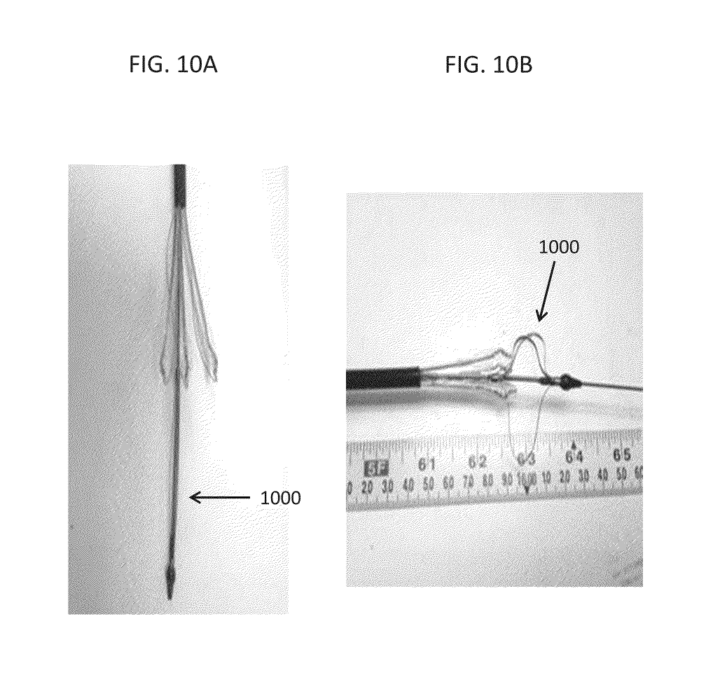

As previously described, FIGS. 9A and 9B show side views of distal treatment portions of shock wave valvuloplasty devices that comprise an anchor 906. As shown, a portion of the anchor has entered the left ventricle 908. The anchor comprises an inner shaft 910 and an outer shaft 912, which may be slidably disposed around a portion of the inner shaft. The expandable portion of the anchor may be one or more arms 914 which are attached distally to a sliding tip 916 of the outer shaft 912. The one or more arms are attached proximally to a portion of the central tubular member 918. With the sliding tip 916 of the outer shaft in a distal position, the one or more arms 914 may be straight and the anchor 906 may be in a low-profile configuration. The sliding tip 916 of the outer shaft 912 may be withdrawn proximally relative to the inner shaft 910, which may withdraw the distal attachment of the arms 914 towards the proximal attachment of the arms while the proximal attachment remains stationary. This may result in the arms 914 bowing out, and the anchor 906 may then be in an expanded configuration. FIGS. 10A and 10B show a distal treatment portion of the shock wave valvuloplasty device with an anchor 1000 in a low-profile and an expanded configuration, respectively. In some variations, the arms may comprise a shape-memory alloy, such as nitinol, which may be pre-formed with a curve. This curve may be the same for each arm of an anchor, such that when an anchor is in an expanded configuration, each arm forms a symmetric curve that bows out from the outer shaft. A preformed curve may be advantageous by preventing kinking of an arm or bending in an undesirable direction. In some variations, the anchor may be at least partially withdrawn into a lumen of the central tubular member during portions of the valvuloplasty procedure.

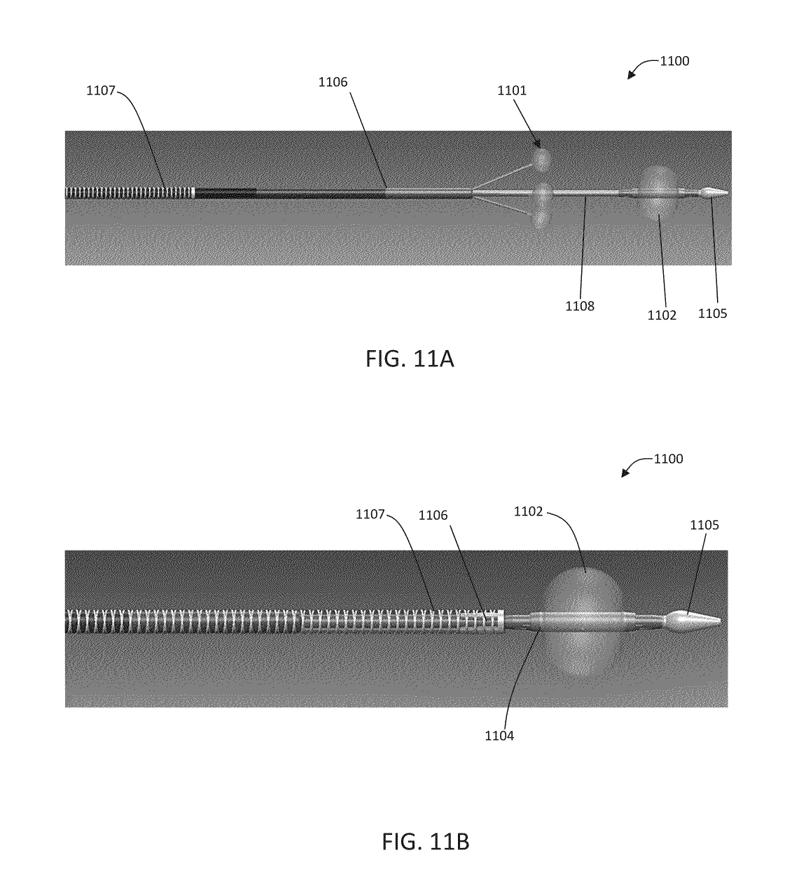

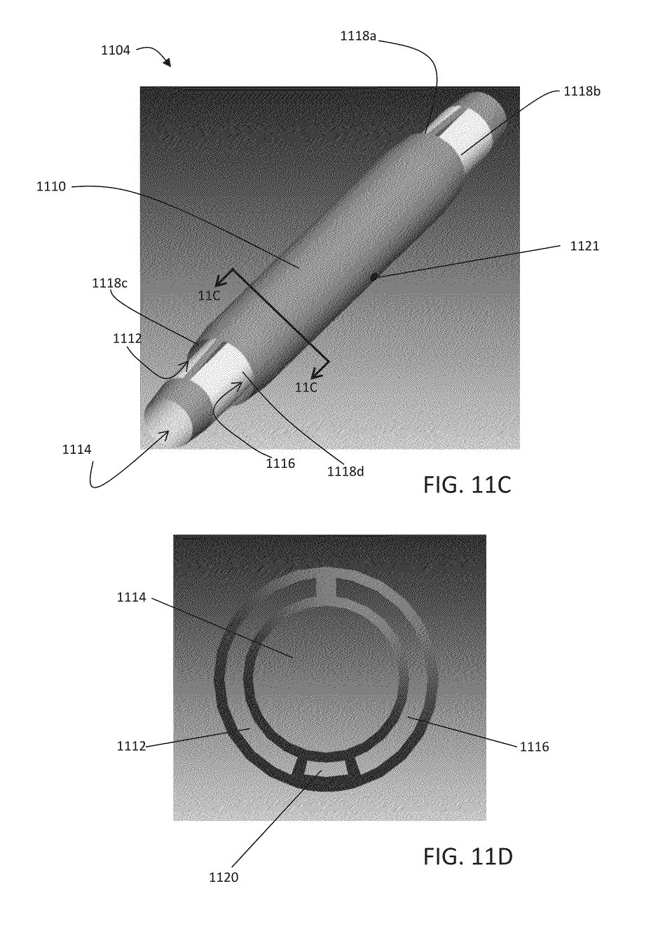

Another variation of an anchor that may be used with any of the shock wave valvuloplasty devices described herein is depicted in FIGS. 11A-11D. FIG. 11A depicts a shock wave valvuloplasty device 1100 that may comprise a main shaft 1106, one or more shock wave balloon catheters 1101 extending from the main shaft 1106, and an anchor that comprises one or more inflatable elastic structures such as a balloon 1102. The anchor may be mounted on an inner shaft 1108 that is slidable within a lumen of the main shaft 1106. Each of the shock wave balloon catheters may comprise one or more pairs of electrodes within the balloon. Although the balloon catheters are depicted as having substantially round, circular balloons, it should be understood that the balloons may have any shape or size, such as the elongated cylindrical balloons described above (e.g., depicts in FIGS. 1A-1C, 9A-9B, etc.). Optionally, the device 1100 may comprise an atraumatic tip 1105 located at the distal-most end of the shaft 1106. The balloon 1102 may have a collapsed configuration and an expanded configuration. The balloon 1102 may be retained in the collapsed configuration within a tubular structure such as an overtube 1107, where retraction of the tube allows for the inflation or expansion of the balloon 1102. The one or more shock wave balloon catheters 1101 may also be compressed in the tube 1107, and retraction of the tube may deploy the shock wave balloon catheters, as depicted in FIG. 11A. After the shock wave valvuloplasty device 1100 has been advanced to the desired position in the vicinity of the valve (e.g., within or through the valve orifice), the balloon 1102 may be inflated with a fluid and assume its expanded configuration. The fluid may be a gas (e.g., helium) and/or liquid (e.g., saline). The balloon 1102 may be inflated via an inflation lumen located on the shaft 1106 (e.g., within the shaft or along its outer surface) in fluid communication with the interior of the balloon.

When the balloon 1102 is inflated as depicted in FIG. 11B, it may have a diameter that approximates the diameter of the valve orifice, and may therefore substantially obstruct or occlude the blood flow through the valve. In some variations, a shock wave valvuloplasty device may comprise a flow lumen or shunt in the vicinity of the balloon anchor so that blood from one side of the valve can flow to the other side when the balloon is expanded. For example, the valvuloplasty device 1100 may comprise a flow diverter 1104 disposed over the surface of the shaft, where a portion of the diverter 1104 extends through the internal cavity of the balloon 1102. In the example depicted in FIGS. 11C-11D, the flow diverter 1104 may comprise a tubular structure 1110 having a first diverter lumen 1112 that extends longitudinally along a length of the tubular structure, and a central lumen 1114 that extends along the entire length of the tubular structure. Optionally, the flow diverter 1104 may comprise a second diverter lumen 1116 that extends longitudinally along a length of the tubular structure that is located opposite the first diverter lumen 1112. The first and second diverter lumens may each have a length that is equal to or greater than the length or diameter of the balloon 1102 so that the openings 1118a,b,c,d of the diverter lumens are located outside of the balloon 1102 (i.e., not enclosed within the balloon). The proximal and distal ends of the balloon anchor 1102 may be sealed over the length of the flow diverter between the openings 1118a,b and 1118c,d. Blood can enter the openings 1118a,b on one side of the first and second diverter lumens and flow across to the other side, exiting through the openings 1118c,d. While two diverter lumens are described in this variation, it should be understood that a flow diverter may have any number of diverter lumens, e.g., 1, 2, 3, 4, 6, 8, 10, 11, 12, etc. The diameter of the central lumen 1114 may correspond with the outer diameter of the shaft 1106. Optionally, the flow diverter 1104 may comprise a balloon anchor inflation lumen 1120 (in addition or alternative to any inflation lumen of the shaft 1106), an example of which is depicted in FIG. 11D. The inflation lumen 1120 may extend along a length of the flow diverter, and may be parallel to the one or more diverter lumens. One or more openings 1121 may be provided on a side wall of the flow diverter that is within the balloon, where the one or more openings 1121 are in fluid communication with the flow diverter inflation lumen 1120. The proximal portion of the inflation lumen may be connected to a proximal fluid source or reservoir via a tube, or a fluid lumen within the shaft 1108, and fluid may be infused or pumped from the source, through the fluid lumen within the shaft 1108, through the inflation lumen 1120, and then through the one or more openings 1121 to inflate the anchor.

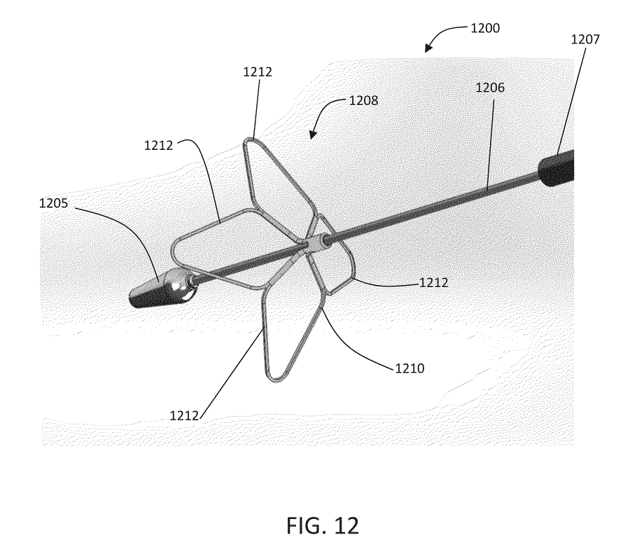

In some variations, a self-expanding anchor may be used so that the anchor may be expanded automatically once it is deployed. One variation of a self-expanding anchor that may be used with any of the valvuloplasty devices described herein is depicted in FIG. 12. A shock wave valvuloplasty device 1200 may comprise a shaft 1206 and an anchor 1208 that comprises a self-expanding scaffold 1210. Optionally, the device 1200 may comprise an atraumatic tip 1205 located at the distal-most end of the shaft 1206. The scaffold 1210 may be made of a shape-memory material such as nickel-titanium alloy. The scaffold 1210 may comprise one or more closed-form structures, such as lobes 1212. The lobes 1212 may be arranged in a radial symmetric configuration around the shaft 1206, or in other variations, may be arranged in a non-symmetric configuration. In some variations, the scaffold 1210 may have a collapsed configuration where the lobes 1212 are compressed against the shaft 1206 and an expanded configuration where the lobes 1212 extend outwardly from the shaft 1206. The scaffold 1210 may be retained in the collapsed configuration within a tubular structure such as an overtube 1207. After the shock wave valvuloplasty device 1200 has been advanced to the desired position in the vicinity of the valve (e.g., within or through the valve orifice), the overtube 1207 may be withdrawn proximally, and thereby allow the scaffold 1210 to expand outwardly. Because the scaffold does not have any walls that would obstruct the blood flow through the valve orifice, a flow diverter may not be included. The number, size and shape of the lobes 1212 may be selected at least in part based on the number, size and shape of the valve leaflets. The angular sweep of each of the lobes around the shaft may also vary depending on the valve geometry. In some variations, the number, size and shape of the lobes may be selected such that the edges of the lobes are not aligned (e.g., counter-aligned) with the intercusp spaces between the leaflets. This may help to resist or prevent the anchor from pulling through the valve orifice after it has been deployed. For example, the anchor 1208 may comprise a scaffold 1210 with four lobes 1212 to resist prevent pulling through a tricuspid valve. The lobes 1212 of the scaffold 1210 may also be configured so that the leaflets and/or cusps can be compressed between the lobes 1212 on one side of the valve and the balloons that enclose the shock wave electrodes on the other side of the valve. In use, the anchor 1208 may be pushed through the valve orifice, expanded, and then pulled up against the valve leaflets to help further engage or contact the shock wave electrode balloons with the leaflets and/or cusps. Optionally, the scaffold 1210 (or the balloon anchor 1102) may comprise one or more shock wave electrodes so that calcifications on both sides of the leaflets may be targeted with the valvuloplasty device.

As shown in FIG. 1A, a shock wave valvuloplasty device 100 may comprise a proximal control 120 that may comprise one or more controls for components at the distal treatment end of the shock wave valvuloplasty device. For example, a proximal control may comprise one or more fluid ports, which may be luer locks, for introducing and withdrawing fluid from one or more balloons individually or simultaneously. A proximal control may comprise one or more controls for advancing and retracting one or more elongated carriers and balloons over one or more positioning wires, individually or simultaneously. In some variations, a proximal control may comprise a control for advancing and retracting an overtube. A proximal control may comprise one or more controls for activating electrode assemblies separately or simultaneously, or any combination of the two (e.g., all electrode assemblies of one balloon simultaneously, but separately from electrode assemblies of another balloon). In some variations, the proximal control may comprise one or more controls for maneuvering an anchor (e.g., a control for advancing the anchor distal to the central tubular member, a control for advancing/retracting the outer shaft to move the anchor between a low-profile and an expanded configuration, a control for rotating the anchor). In some variations, the shock wave valvuloplasty device may comprise a guidewire, which may be the inner shaft of the anchor. A proximal control may comprise a control or port for manipulating the guidewire.

### Methods

Methods for use of a shock wave valvuloplasty device to treat aortic heart valve tissue will be described here. Generally, these methods comprise introducing a shock wave valvuloplasty device into a patient's vasculature and advancing the device into proximity of the aortic valve. The device may then be aligned with the cusps of the aortic valve, which may comprise positioning an inflatable balloon adjacent to each cusp of the aortic valve. Alignment may be facilitated by advancing a positioning wire distal bend into contact with each valve cusp and/or deploying an expandable anchor through the aortic valve and into the left ventricle. Optionally, after the anchor has been deployed and expanded, the anchor may be pulled up against the underside of the leaflets such that the leaflets are pressed between the anchor and the balloon of the shock wave valvuloplasty device. Once the shock wave valvuloplasty device is aligned as desired, shock waves may be delivered to one valve cusp at a time or to more than one valve cusp simultaneously. Delivering shock waves to a cusp may comprise delivering high voltage pulses to electrode assemblies positioned on an elongated carrier within an inflated balloon. The high voltage pulses may generate shock waves originating from each electrode assembly that may propagate through the fluid filled balloon and be transferred to an adjacent valve cusp in order to break up calcified lesions. The direction the shock waves are propagated, and the portion of the valve that is targeted, may be controlled in one or more ways. For example, an elongated carrier, balloon, and shock wave assemblies may be advanced and/or retracted over a positioning wire. The positioning wire may comprise one or more curves or kinks, which may cause shock waves to be propagated in different directions. An overtube may be advanced over a portion of the positioning wire, which may move the positioning wire, elongated carrier, balloon, and electrode assemblies towards an inner portion of the valve. After delivery of shock waves to each cusp of the aortic valve, each balloon may be deflated, the anchor returned to a low-profile configuration, and the shock wave valvuloplasty device withdrawn through the vasculature and out of the patient.

The methods described here may be performed while practicing sterile technique. A patient's vasculature may be accessed and at least a distal treatment portion of the shock wave valvuloplasty device may be introduced into the vasculature. The shock wave valvuloplasty device may be advanced in a retrograde fashion to the proximity of the aortic valve, which in some variations may be facilitated by advancement of the shock wave valvuloplasty device over a guidewire. Once in proximity to the aortic valve, the shock wave valvuloplasty device may be aligned with one or more cusps of the aortic valve. Alignment may comprise positioning one or more balloons of the shock wave valvuloplasty device within the concave portion of one or more aortic valve cusps. It should be appreciated that while a balloon may contact a valve cusp, it need not. Energy from shock waves may still be delivered to calcified tissue across a gap between a balloon and a valve cusp. In some variations, a shock wave valvuloplasty device may comprise two balloons, such as for use in patients with a bicuspid aortic valve. In other variations, a shock wave valvuloplasty device may comprise three balloons, such as for use in patients with a tricuspid aortic valve.

Positioning a balloon in the cusp of an aortic valve may be facilitated by a distal bend in a positioning wire. In some variations, a distal bend may be the most distal portion of the shock wave valvuloplasty device. A user may advance the shock wave valvuloplasty device to an aortic valve until a distal bend of a positioning wire engages an aortic valve cusp. At this point, a user may sense resistance to further advancement of the device, indicating that the delivery device is at a desired longitudinal position relative to the aortic valve. In some variations of the methods, all of the one or more balloons may be inflated when the shock wave valvuloplasty device is in the proximity of the aortic valve, which may facilitate self-alignment of each balloon in a valve cusp.

In some variations, an anchor may facilitate alignment of each balloon with a valve cusp. An anchor may comprise one or more arms that may extend radially from an outer shaft of the anchor. In some variations, the arms may be circumferentially oriented to align with the spaces between each valve cusp (intercusp spaces). The anchor may be prevented from crossing the aortic valve unless each arm is oriented with an intercusp space. Each balloon may be circumferentially oriented halfway between each arm, which may align each balloon with a valve cusp when each arm is oriented with an intercusp space. A user may advance and rotate the shock wave valvuloplasty device until it crosses the aortic valve, which may indicate that the arms of the anchor have aligned with each intercusp space and the balloons have aligned with each valve cusp.

The anchor may be movable between a low-profile configuration and an expanded configuration. In an expanded configuration, the arms may extend farther from the outer shaft of the anchor than they do in a low-profile configuration. After crossing the aortic valve into the left ventricle, the anchor may be expanded. This may hold the distal treatment portion of the shock wave valvuloplasty device in a desired position for directive shock waves to the aortic valve area. A user may move the anchor from a low-profile configuration to an expanded configuration by using a proximal control to proximally retract an outer shaft of the anchor relative to an inner shaft of the anchor. A distal portion of one or more arms may be attached to the outer shaft and be movable, whereas a proximal portion of the one or more arms may be attached to a central tubular member and be stationary. Proximal retraction of the outer shaft proximally withdraws the distal attachment of the arms towards the proximal attachment of the arms, which may cause the arms to bow out from the outer shaft to an expanded configuration. While one or more distal bends, one or more balloons, and an anchor may facilitate self-alignment of the shock wave valvuloplasty device, one or more portions of the shock wave valvuloplasty device may be radiopaque such that fluoroscopy may be used for confirmation of position. Alternatively, a valvuloplasty device with an inflatable or self-expandable anchor may be advanced such that the anchor crosses the aortic valve into the left ventricle. Once the anchor is within the ventricle or below the aortic valve leaflets, fluid may be introduced to the inflatable anchor to expand the anchor. Alternatively, where a self-expanding anchor is used, a sheath compressing the anchor may be withdrawn proximally, thereby allowing the anchor to self-expand. After the expansion of any of the anchors described above, the shaft may be pulled proximally to seat the anchor structures against the underside of the leaflets such that the leaflets are clamped between the anchor structures and the shock wave balloons.

Once the shock wave valvuloplasty device is aligned with the aortic valve cusps, shock waves may be delivered to the aortic valve. A first step may be to inflate one or more balloons within the one or more cusps that will be treated. In some variations, more than one balloon may be inflated and/or deliver shock waves simultaneously. In some variations, it may be desirable to have only one balloon inflated at a time and to treat one cusp at a time. This may minimize the resistance to movement of the valve cusps not being treated and minimize any decrease in cardiac output that may result from restriction of valve cusp movement. In some variations, the shock wave valvuloplasty device may comprise one balloon, and the balloon may facilitate treatment of each valve cusp, one after another. In some variations, a shock wave valvuloplasty device may comprise two or three balloons, and only one balloon may be inflated at a time during treatment of a cusp. After treatment of a cusp, the inflated balloon within that cusp may be deflated and a next balloon may be inflated to treat a next cusp. Balloons may be inflated and deflated with a fluid simultaneously or separately through introduction and withdrawal, respectively, of a fluid from one or more ports of a proximal control. The one or more balloons may be fluidly connected with these one or more ports by one or more fluid channels.

An inflated balloon may be positioned such that shock waves generated within the balloon are maximally transferred to a desired portion of a valve cusp. One or more electrode assemblies may be positioned on an elongated carrier within each balloon. The elongated carrier, and thus the electrode assemblies and balloon, are slidably disposed over a portion of a positioning wire associated with each elongated carrier. A balloon may be distally advanced or proximally retracted over a positioning wire to move the balloon wall into the proximity of a valve cusp and/or to change the position of an electrode assembly on the positioning wire. Changing the position of an electrode assembly may change the direction of shock waves that are generated, as was discussed in more detail above. The electrode assemblies may be advanced and retracted over a straight portion of a positioning wire to longitudinally affect the direction of shock wave propagation. The electrode assemblies may be advanced and retracted over a kinked portion of a positioning wire, which may angle the direction of shock wave propagation (e.g., angle the direction of shock wave generation inferiorly towards a cusp). Some variations of a shock wave valvuloplasty device may comprise an overtube, which may be advanced over a portion of a positioning wire to radially displace the positioning wire inwards. This may accordingly radially displace the elongated carrier slidably disposed over the positioning wire, and the balloon and electrode assemblies attached to the elongated carrier.

In some variations of the anchor, the arms or lobes may be circumferentially aligned with each balloon and valve cusp, and not with the intercusp spaces. This may position a valve cusp between a balloon and an anchor arm when the anchor is in the expanded configuration. This configuration may allow shock waves generated on the concave side of a valve cusp to be reflected on the convex side of the valve cusp by an anchor arm, which may enhance cracking or softening calcified tissue in the cusp. In some variations, a user may rotate the arms around a central axis of the anchor between a first, alignment position with the anchor arms offset from the balloons and a second, treatment position with the anchor arms aligned with the balloons. In these variations, a user may advance an anchor across the aortic valve in the alignment position in order to align each anchor arm with each intercusp space, and align each balloon with the center of each valve cusp. Prior to treatment, a user my rotate the anchor into the treatment position in order align the arms with the balloons and facilitate reflection of shock waves.

After a balloon has been positioned in such a way as to deliver shock waves to a desired portion of a valve, high-voltage pulses may be delivered to electrode assemblies within the balloon. The way in which high-voltage pulses are sent to each electrode assembly may be determined, at least in part, by the system of wiring of the electrode assemblies. For example, an electrode assembly may comprise more than one inner electrode, and the more than one inner electrode may be wired in series. In some variations, each inner electrode may be connected to a separate voltage channel in a direct connect configuration, such that shock waves originating from each inner/outer electrode pair may be activated separately. Similarly, more than one electrode assembly may be positioned on an elongated carrier. Each electrode assembly (e.g. the inner and outer electrode pairs on an electrode assembly) may be controlled separately or simultaneously. In some variations, it may be advantageous for shock wave generators to be connected in series and simultaneously activated, as this may decrease the time of the valvuloplasty procedure. In some variations it may be desirable to control shock wave generators independently in order to target specific portions of the valve area.

After all desired portions of the valve area have been targeted with shock waves, all balloons may be deflated. An anchor may be returned to a low-profile configuration, by advancing an outer shaft of the anchor distally in order to straighten one or more bowed arms. The anchor may then be withdrawn through the aortic valve and out of the left ventricle, and the shock wave valvuloplasty device may be withdrawn from a patient's vasculature. Either before or after the shock wave valvuloplasty device is completely withdrawn from the vasculature, a user may determine the mechanical and/or physiologic changes associated with the procedure by any suitable method (e.g., transesophageal echocardiogram, fluoroscopy, measurement of ejection fraction). In some variations, if improvements are less than desired, one or more portions of the valvuloplasty procedure may be repeated.

1. A valvuloplasty device for treating aortic leaflets comprising:
a central tubular member carrying at least two balloon catheters, each balloon catheter including central carrier having a shock wave generator, each balloon catheter further including a balloon affixed to the distal end of the carrier, said balloon being inflatable with a conductive fluid, and wherein activation of each of the shock wave generators creates a shock wave that propagates through the associated balloon; and
a central anchor extending between and beyond the ends of the balloons and configured to pass through the aortic leaflets and into the ventricle to stabilize the position of the balloon catheters.

2. The valvuloplasty device of claim 1, wherein each balloon catheter further comprises a wire extending from the distal end of the balloon, said wire including a U-shaped section configured to center and position the balloon within a cusp of a valve leaflet.

3. The valvuloplasty device of claim 2, wherein a length of the wire extends within the interior of the balloon, and the carrier is slidable over the wire within the balloon such that sliding the carrier over the wire changes the location of the shock wave generator.

4. The valvuloplasty device of claim 3, wherein the length of the wire within the balloon has a bend.

5. The valvuloplasty device of claim 4, wherein the carrier is slidable over the bend.

6. The valvuloplasty device of claim 1, wherein the central anchor comprises an inflatable member disposed over a distal portion of the central tubular member.

7. The valvuloplasty device of claim 6, further comprising a flow diverter disposed over the distal portion of the central tubular member, the flow diverter comprising a proximal opening, a distal opening, and a lumen extending therebetween, the flow diverter arranged such that the proximal and distal openings are located outside of the inflatable member.

8. The valvuloplasty device of claim 1, wherein the central anchor is a self-expanding anchor.

9. The valvuloplasty device of claim 8, wherein the central anchor comprises a compressible scaffold structure.

10. The valvuloplasty device of claim 8, wherein the central anchor comprises a compressible cage.

11. The valvuloplasty device of claim 1, wherein the shock wave generator of each of the balloon catheters comprise a pair of electrodes, and wherein when said pair of electrodes is connected to a high voltage source, a plasma arc is created across the electrodes resulting in a shock wave that propagates through the associated balloon.

12. The valvuloplasty device of claim 1, wherein the shock wave generator of each of the balloon catheters comprises a laser light source.

13. The valvuloplasty device of claim 8, wherein the central anchor comprises a shape-memory material.

14. A valvuloplasty device comprising:
an elongated hollow carrier, said carrier including at least one pair of electrodes;
a balloon affixed to the distal end of the carrier, said balloon being inflatable with a conductive fluid, and wherein said pair of electrodes are located within the balloon and when said pair of electrodes are connected to a high voltage source, a plasma arc is created across the electrodes resulting in a shock wave that propagates through the balloon; and
a positioning wire, slidably received within the carrier, said positioning wire having a bend formed along a length of the wire that is located within the balloon and arranged so that when the position of the carrier is adjusted with respect to the positioning wire, the bend in the wire varies the angular orientation of the pair of electrodes to adjust the propagation direction of the shock wave.

15. The valvuloplasty device of claim 14, wherein the positioning wire extends from the distal end of the balloon, said positioning wire including a U-shaped section configured to center and position the balloon within a cusp of a valve leaflet.

16. The valvuloplasty device of claim 14, further comprising an elongate member and an anchor disposed over a distal portion of the elongate member, wherein the elongate member is configured to extend distally beyond the balloon to pass the anchor through a valve orifice to stabilize the position of the balloon.

17. The valvuloplasty device of claim 16, wherein the anchor comprises an inflatable member.

18. The valvuloplasty device of claim 17, further comprising a flow diverter disposed over the distal portion of the elongate member, the flow diverter comprising a proximal opening, a distal opening, and a lumen extending therebetween, the flow diverter arranged such that the proximal and distal openings are located outside of the inflatable member.

19. The valvuloplasty device of claim 16, wherein the anchor is a self-expanding anchor.

20. The valvuloplasty device of claim 19, wherein the anchor comprises a compressible scaffold structure.

21. The valvuloplasty device of claim 19, wherein the anchor comprises a compressible cage.

22. The valvuloplasty device of claim 19, wherein the anchor comprises a shape-memory material.

23. A shock wave valvuloplasty method comprising:
advancing a shock wave valvuloplasty device to an aortic valve, the device comprising a central tubular member carrying at least two balloon catheters, each balloon catheter including central carrier having a shock wave generator, each balloon catheter further including a balloon affixed to the distal end of the carrier, and a central anchor extending between and beyond the ends of the balloons;
advancing the central anchor through the aortic leaflets and into the ventricle;
deploying the central anchor within the ventricle to stabilize the position of the balloon catheters;
inflating the balloon of at least one of the balloon catheters with a conductive fluid to seat the at least one balloon within the cusp of an aortic leaflet; and
activating the shock wave generator to initiate one or more shock waves.

24. The method of claim 23, wherein deploying the central anchor comprises expanding the anchor from a compressed delivery configuration to an expanded deployed configuration.

25. The method of claim 24, wherein the central anchor comprises an anchor balloon.

26. The method of claim 24, wherein the central anchor comprises a shape-memory scaffold structure.

27. The method of claim 23, wherein the shock wave generator of each of the balloon catheters comprises a pair of electrodes and wherein activating the shock wave generator comprises applying a high voltage across said pair of electrodes such that a plasma arc is created across the electrodes resulting in a shock wave that propagates through the associated balloon.

28. The method of claim 23, wherein the shock wave generator of each of the balloon catheters comprises a laser light source, and wherein activating the shock wave generator comprises generating laser light of sufficient energy to generate a shock wave that propagates through the associated balloon.

29. The method of claim 23, further comprising clamping the aortic leaflet between an inflated balloon of at least one of the balloon catheters and the deployed central anchor.

30. A shock wave valvuloplasty method comprising:
advancing a shock wave valvuloplasty device to an aortic valve, the device comprising an elongated hollow carrier, said carrier including at least one pair of electrodes, a balloon affixed to the distal end of the carrier and wherein the at least one pair of electrodes are located within the balloon, and a positioning wire slidably received within the carrier, said positioning wire having a bend formed along a length of the wire that is located within the balloon;
inflating the balloon with a conductive fluid to seat the at least one balloon within the cusp of an aortic leaflet;
adjusting the carrier with respect to the positioning wire, wherein the bend in the wire varies the angular orientation of the pair of electrodes and thereby adjusts the propagation direction of the shock wave; and
applying a high voltage across said pair of electrodes such that a plasma arc is created across the electrodes resulting in a shock wave that propagates at a first direction from a first location through the balloon.

31. The method of claim 30, further comprising moving the carrier with respect to the positioning wire to adjust the angular orientation and location of the pair of electrodes and applying a high voltage across said pair of electrodes such that a plasma arc is created across the electrodes resulting in a shock wave that propagates at a second direction from a second location through the balloon that is different from the first direction and the first location.
